(19) United States
(12) Patent Application Publication
(10) Pub. No.: US 2012/0253046 A1
(43) Pub. Date: Oct. 4, 2012

(54) PALONOSETRON METABOLITES

(75) Inventor: Waldo Mossi, Mendrisio (CH)

(73) Assignee: Helsinn Healthcare S.A., Lugano (CH)

(21) Appl. No.: 13/501,171

(22) PCT Filed: Nov. 1, 2010
(86) PCT No.: PCT/IB2010/002893

§ 371 Date: Jun. 19, 2012

Related U.S. Application Data
(60) Provisional application No. 61/260,916, filed on Nov. 13, 2009.

(51) Int. Cl. C07D 471/08

(52) U.S. Cl. 546/99

(57) ABSTRACT

Provided are metabolites of palonosetron that can be used in treating animals, particularly humans, of the formula (I): or a pharmaceutically acceptable salt or prodrug thereof; wherein R¹ and R⁴ independently can be H, hydroxyl, or carbonyl; and wherein R³ can be Formule (II) or Formule (III).

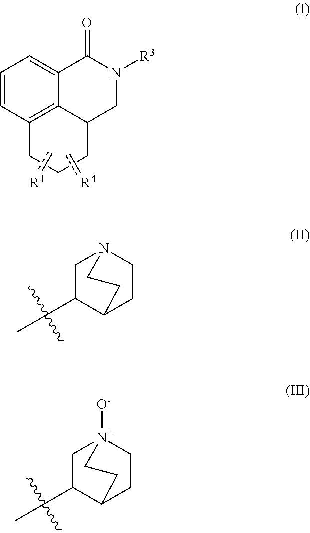

### RELATION TO PRIOR APPLICATIONS

This application claims priority to U.S. Provisional Application 61/260,916, filed Nov. 13, 2009.

### FIELD OF THE INVENTION

The present invention relates to metabolites of palonosetron.

### BACKGROUND OF THE INVENTION

The nausea and emetogenic side effects of anti-cancer chemotherapy and radiotherapy are a widespread and longstanding problem. Perhaps less well known but no less important are post-operative nausea and emesis, which may have physiological mechanisms related to the effects seen for chemotherapy.

Palonosetron hydrochloride has recently emerged as a highly efficacious anti-nauseant and anti-emetic against these conditions. See PCT publications WO 2004/045615 and 2004/073714 from Helsinn Healthcare. Palonosetron hydrochloride is sold in the United States as a sterile injectable liquid under the ALOXI® brand, in sterile unit dose vials containing 0.075 or 0.25 mg. of palonosetron hydrochloride. Palonosetron hydrochloride also is also sold as an orally administered soft-gel dosage form containing 0.5 mg. of palonosetron hydrochloride.

The official chemical name for palonosetron hydrochloride is (3aS)-2-[(S)-1-Azabicyclo[2.2.2]oct-3-yl]-2,3,3a,4,5,6-hexahydro-1-oxo-1Hberiz[de]isoquinoline hydrochloride (CAS No. 119904-90-4); its empirical formula is C₁₉H₂₄N₂O.HCl, and its molecular weight is 332.87. The compound is represented by the following chemical structure:

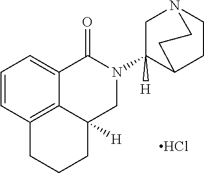

Methods of synthesizing palonosetron are described in U.S. Pat. Nos. 5,202,333 and 5,510,486. Pharmaceutically acceptably dosage forms are described in PCT publications WO 2004/067005 and WO 2008/049552 from Helsinn Healthcare.

### SUMMARY OF THE INVENTION

The present invention is premised on the discovery that palonosetron metabolizes into novel compounds when administered to mammals. Based on these discoveries, metabolites have been synthesized that exhibit utility in treating animals, particularly humans.

Thus, in one embodiment the invention provides a compound comprising formula I:

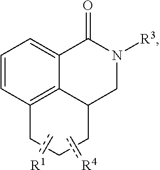

or a pharmaceutically acceptable salt or prodrug thereof;

wherein R¹ and R⁴ independently can be H, hydroxyl, or carbonyl; and

wherein R³ can be

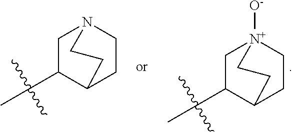

In some aspects of the invention, R¹ and R⁴ can independently be in the 4, 5, or 6 position. A person skilled in the art would recognize that if R¹ is a carbonyl, then R⁴ would not occupy the same position. Also, a person skilled in the art would recognize that if R⁴ is a carbonyl, then R¹ would not occupy the same position.

Additional embodiments and advantages of the invention will be set forth in part in the description which follows, and in part will be obvious from the description, or may be learned by practice of the invention. The embodiments and advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF THE INVENTION

The present invention may be understood more readily by reference to the following definitions and detailed description of preferred embodiments of the invention and the non-limiting Examples included therein.

### Definitions and Use of Terms

As used in this specification and in the claims which follow, the singular forms “a,” “an” and “the” include plural referents unless the context clearly dictates otherwise. Thus, for example, reference to “an ingredient” includes mixtures of ingredients, reference to “an active pharmaceutical agent” includes more than one active pharmaceutical agent, and the like.

Throughout the description and claims of this specification, the word “comprise” and variations of the word, such as “comprising” and “comprises,” means “including but not limited to,” and is not intended to exclude, for example, other additives, components, integers or steps.

The terms “treating” and “treatment,” when used herein, refer to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use. “Pharmaceutically acceptable salts” means salts which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2,-ethanedisulfonic acid, 2-hydroxyethanesulfonic acid, benzenesulfonic acid p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

In addition, pharmaceutically acceptable salts may be formed when an acidic proton present is capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

“Leaving group” has the meaning conventionally associated with it in synthetic organic chemistry, i.e., an atom or group displaceable under alkylating conditions, and includes halogen and alkane- or arenesulfonyloxy such as mesyloxy, ethanesulfonyloxy, benzenesulfonyloxy, tosyloxy and the like.

Compounds that have identical molecular formulae but differ in the nature or sequence of bonding of their atoms or in the arrangement of their atoms in space are termed “isomers.” Isomers that differ in the nature or sequence of bonding of their atoms are termed “constitutional isomers.” Isomers that differ only in the arrangement of their atoms in space are termed “stereoisomers.” Stereoisomers that are not mirror images of one another are termed “diasteromers” and stereoisomers that are mirror images are termed “enantiomers” or sometimes “optical isomers.” Stereoisomers that are superimposable upon their mirror images are termed “achiral” and those not superimposable are termed “chrial.” A carbon atom bonded to four different groups is termed a “chiral center” or alternatively an “asymmetric carbon.”

When a compound has a chiral center, a pair of enantiomers of opposite chirality is possible. An enantiomer can be characterized by the absolute configuration of its chiral center and described by the R- and S-sequencing rules of Cahn and Prelog (i.e., as (R)- and (S)-isomers) or by the manner in which the molecule rotates the plane of polarized light and designated as dextrorotatory or levorotatory (i.e., as (+)- and (−)-isomers, respectively). A chiral compound can exist as either individual enantiomer or as a mixture thereof. A mixture containing equal proportions of the enantiomers is termed a “racemic mixture” or “racemate” and may be described as the (RS)- or (+−)-mixture thereof.

Unless indicated otherwise, the description or naming of a particular compound in the specification and claims is intended to include both individual enantiomers and mixtures, racemic or otherwise, thereof. Conventions for stereochemical nomenclature, methods for the determination of stereochemistry and the separation of stereoisomers are well-known in the art (see discussion in Chapter 4 of “Advanced Organic Chemistry”, 3rd edition March, Jerry, John Wiley and Sons, New York, 1985).

Certain compounds of Formulae I and XI can exist as stereoisomers. For example, certain compounds possess a chiral center at the ring carbon of the R³ substituent which is bonded to the amide nitrogen and, when the optional bond is absent, at the 3a-position and therefore can exist as (R)- or (S)-isomers. In addition, certain compounds can exist as the (endo)- or (exo)-isomers, e.g., when the R³ substituent is 1-azabicyclo[3.3.1]non-4-yl.

When a compound of Formula I or XI possesses one chiral center, a pair of enantiomers exists. When two chiral centers are present in a compound of Formula II, four separate steroisomers exist (i.e., two separate pairs of enantiomers). When a compound of Formula II possesses two chiral centers and can exist as endo or exo, eight separate stereoisomers are possible (i.e., two separate pairs of enantiomers in the endo or exo form).

It is to be understood that when referring to Formula I, Ia, XI and XIa in this application, a straight line depicting the covalent bond between the R³ substituent and the amide nitrogen represents the possible geometric isomers and enantiomers or the mixtures, racemic or otherwise, thereof. Similarly, when referring to Formula II in which the optionally bond is absent, a straight line depicting the covalent bond between carbons 3a and 4 represents either the R or S configurations or a mixture racemic, or otherwise, thereof. For purposes of the present application when referring to a compound by name or by formula and the configuration is not designated, it is to be understood that the reference is to all possible forms.

### Metabolites

In one embodiment the invention provides a compound comprising the Formula I:

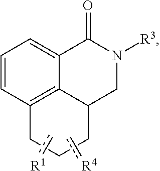

or a pharmaceutically acceptable salt thereof;

wherein R¹ and R⁴ independently can be H, hydroxyl, or carbonyl; and

wherein R³ can be

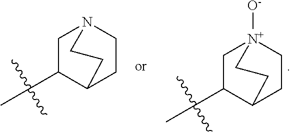

In some aspects of the invention, R¹ and R⁴ can independently be in the 4, 5, or 6 position. A person skilled in the art would recognize that if R¹ is a carbonyl, then R⁴ would not occupy the same position. Also, a person skilled in the art would recognize that if R⁴ is a carbonyl, then R¹ would not occupy the same position.

In some aspects of the invention, Formula I can be optically pure.

In some aspects of the invention, R¹ and R⁴ can independently be either R or S enantiomers.

In some aspects of the invention, R¹ can be a hydroxyl group in the R form in the 6 position.

In some aspects of the invention, R¹ can be a hydroxyl group in the S form in the 6 position.

In some aspects of the invention, R¹ can be a hydroxyl group in the R form in the 5 position and R⁴ can be a hydroxyl group in the S form in the 6 position.

In some aspects of the invention, R³ can be in the S form.

In some aspects of the invention, R¹ can be a carbonyl and R⁴ can be H.

In some aspects of the invention, R¹ can be a carbonyl in the 6 position.

In some aspects of the invention, R¹ and R⁴ can be H.

In another embodiment the invention provides a compound comprising formula II:

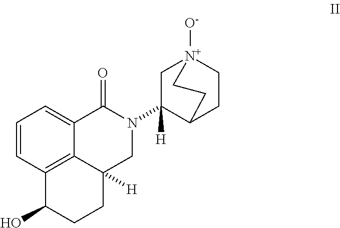

or a pharmaceutically acceptable salt or prodrug thereof.

In another embodiment the invention provides a compound comprising formula III:

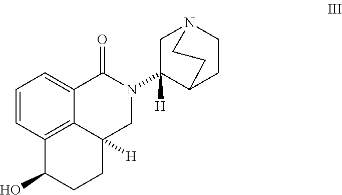

or a pharmaceutically acceptable salt or prodrug thereof.

In another embodiment the invention provides a compound comprising formula IV:

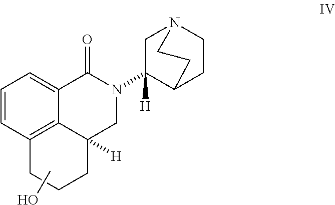

or a pharmaceutically acceptable salt or prodrug thereof.

In another embodiment the invention provides a compound comprising formula V:

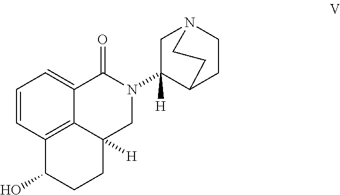

or a pharmaceutically acceptable salt or prodrug thereof.

In another embodiment the invention provides a compound comprising formula VI:

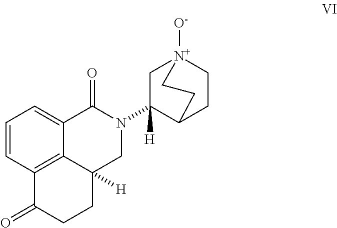

or a pharmaceutically acceptable salt or prodrug thereof.

In another embodiment the invention provides a compound comprising formula VII:

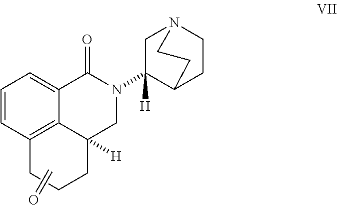

or a pharmaceutically acceptable salt or prodrug thereof.

In another embodiment the invention provides a compound comprising formula VIII:

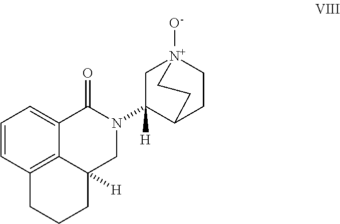

or a pharmaceutically acceptable salt or prodrug thereof.

In another embodiment the invention provides a compound comprising formula IX:

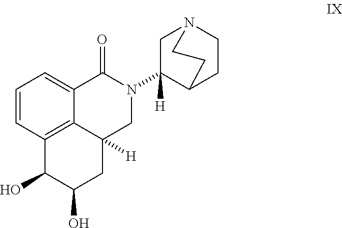

or a pharmaceutically acceptable salt or prodrug thereof.

### Processes for Preparing Compounds of the Invention

Compounds of Formula I can be prepared by the reaction sequence shown below in Reaction Scheme I.

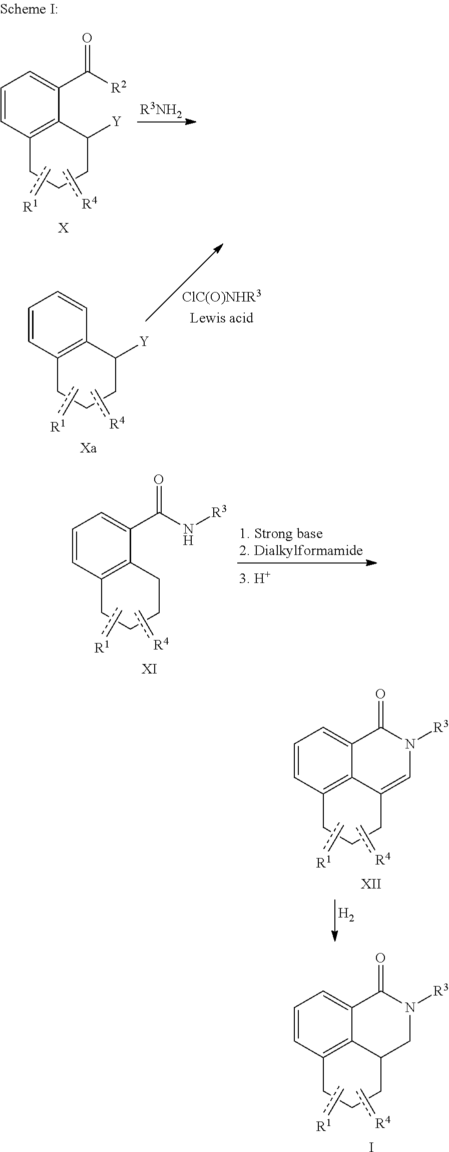

in which R² is hydroxy, alkoxy or halogen and Y is hydrogen or R² and Y together are oxa, and R¹ and R⁴ independently are H, hydroxyl, or carbonyl.

R¹ and R⁴ can independently be ortho, meta, or para to Y in X and Xa. A person skilled in the art would recognize that if R¹ is a carbonyl, then R⁴ would not occupy the same position relative to Y. Also a person skilled in the art would recognize that if R⁴ is a carbonyl, then R¹ would not occupy the same position relative to Y.

Compounds of Formula I are conveniently prepared by a two step synthesis comprising (1) converting an acid or acid derivative of Formula X or a fused-ring bicyclic compound of Formula Xa to a substituted amide of Formula XI and (2) reacting the amide with a formylating agent in the presence of a strong base and then acidifying to form a compound of Formula XII (a compound in which the optional bond is present). Compounds of Formula I (compounds in which the optional bond is absent) are subsequently prepared by reduction.

During the synthesis of Formula I, a person skilled in the art would recognize when and if the use of protection groups would be necessary. For instance, if R¹ and R⁴ would be hydroxyl groups a person skilled in the art would know when and how to protect such group during the course of the synthesis of Formula I. Techniques and non limiting examples of protecting groups can be found in Michael B. Smith, Organic Synthesis 2^{nd} Ed. McGraw-Hill Higher Education, New York, 2002.

Step 1

Compounds of Formula XI are prepared by reacting a compound of Formula X with a substituted amine of the formula NH₂R³ in which R³ is

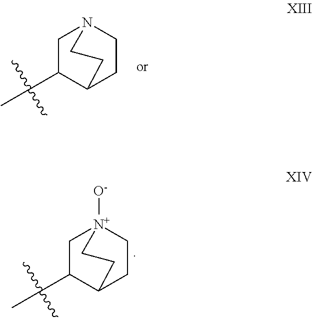

Reaction conditions are those standard for amide formation (e.g., see J. Advanced Organic Synthesis March 1985, 3rd Ed., 370-376). Generally the reaction is carried out at 20° C. to 200° C., preferably −10° C. to 20° C., and ambient pressure for 0.5 to 3 hours in a suitable inert organic solvent (e.g., methylene chloride, THF and toluene).

Alternatively, compounds of Formula XI may be prepared by Friedel-Crafts acylation in which a chloroformamide of the formula ClC(O)NHR³ is reacted with a compound of Formula Xa in the presence of a Lewis acid such as aluminum chloride, boron trifluoride, hydrogen fluoride or phosphoric acid.

In general, the starting materials utilized in the preparation of compounds of Formula XI are known to or can readily be synthesized by those of ordinary skill in the art. For example, similar compounds of Formula X are discussed by Lowenthal, H. J.; Schatzmiller, S. J. Chem. Soc. Perkin Trans. I 1976, 944. Unsubstituted compounds are readily available or may be prepared in accordance with methods known in the art.

Compounds of Formula X wherein R² and Y are together oxa can be prepared from an alcohol of the formula

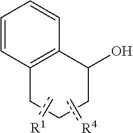

by treating with a strong base (e.g., n-butyllithium) in an inert organic solvent (e.g., hexanes) for approximately 20 hours followed by bubbling through with carbon dioxide for approximately 5 hours.

Other starting materials that are useful for preparing compounds of the invention are 1-cyano-4-alkoxynaphthalenes which can be hydrolyzed and reduced to the corresponding starting acid of Formula X wherein R² is hydroxy, Halogen-substituted tetralones are well known and are prepared from o-halophenylbutyric acids. These tetralones can be reduced to the appropriate alcohol, converted to an acid and reacted with the R³NH₂ compound as a lactone to form an amide of Formula XI.

Step 2

Compounds of Formula XII are prepared by reacting amides of Formula XI with a dialkylformamide in the presence of a strong base and than acidifying. The reaction is carried out in a inert ethereal solvent (e.g., diethyl ether, dimethoxyethane or tetrahydrofuran (THF), preferably THF) at temperatures ranging from −70° C. to 25° C., preferably −20° C. to 0° C., at ambient pressure and under an inert atmosphere (e.g., argon or nitrogen, preferably nitrogen). The dialkylformamide, preferably dimethylformamide (DMF), is generally used in molar excess relative to the amide of Formula XI. Any strong base, such as a Grignard reagent or an appropriate alkyllithium, preferably n-butyllithium, can be utilized.

Compounds of Formula I may be prepared by reduction of the corresponding compound of Formula XII. The reduction is carried out under standard hydrogenation conditions with an appropriate hydrogenation catalyst and in a suitable polar, organic solvent. Reaction pressures may vary from atmospheric to approximately 15 megaPascals (mPa) and temperatures may range from ambient to approximately 100° C. While any standard catalyst (e.g., rhodium on alumina, etc.) may be used, certain catalysts are preferred. Preferred catalysts include 10% palladium hydroxide, 20% palladium hydroxide on carbon, Pearlman's catalyst (50% H₂O-20% palladium content) and palladium/BaSO₄. Suitable solvents include ethanol, DMF, acetic acid, ethyl acetate, tetrahydrofuran, toluene, and the like.

Depending upon the catalyst, solvent, pressure and temperature chosen, the reduction process may take from a few hours to a few days to complete. As an example, a reaction carried out with 20% palladium hydroxide in acetic acid and 70% perchloric acid at 15 kPa and 85° C. takes approximately 24 hours for full reduction to occur.

A compound of Formula XII can be reduced in either the nonsalt or salt form. If an optically active reagent is employed to form the salt of a compound of Formula XII, formation of one enantiomer over the other may be favored.

Compounds of Formula XII and I are also prepared by the reaction sequence shown below in Scheme II.

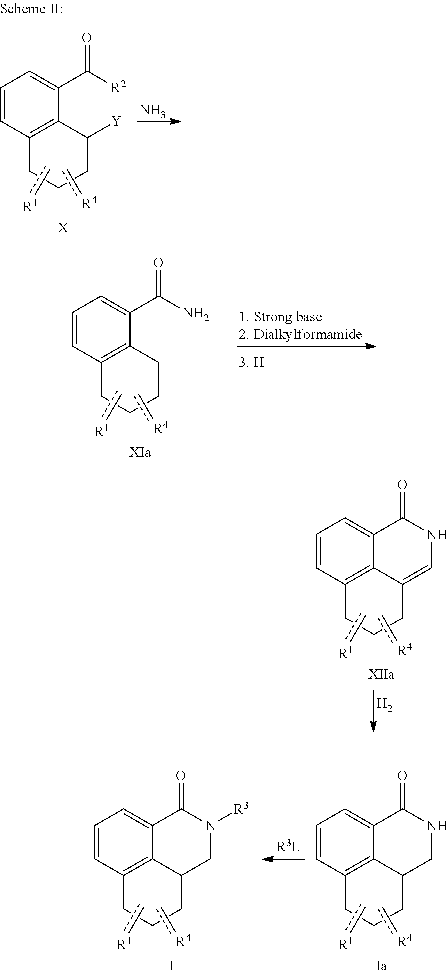

in which R² is hydroxy, alkoxy or halogen and Y is hydrogen or R² and Y together are oxa, L is a leaving group, R¹, R⁴ and R³ are as defined elsewhere herein.

Alternatively, compounds of Formula XII and I are prepared by a three step synthesis comprising (1) converting an acid or acid derivative of Formula A to an unsubstituted amide of Formula XIa, (2) reacting the amide with a formylating agent in the presence of a strong base and then acidifying to form a compound Formula XIIa (a compound of Formula XII in which the optional bond is present), (3) optionally reducing a compound of Formula XIIa to a compound of Formula Ia (a compound of Formula I in which the optional bond is absent) and (4) condensing the compound of Formula Ia with an appropriate alkylating agent to form a compound of Formula I.

Step 1

Compounds of Formula XIa are prepared by proceeding as in Step 1 of Scheme I but replacing the substituted amine with ammonia.

Step 2

Compounds of Formula XIIa are prepared by proceeding as in Step 2 of Scheme I but substituting a compound of Formula XIa for the compound of Formula XI. Compounds of Formula XIIa may be prepared by proceeding as described above for the hydrogenation of a compound of Formula XII but substituting a compound of Formula XIIa.

Step 3

Compounds of Formula I are prepared by reacting, in the presence of a strong base, a compound of Formula XIIa with an alkylating agent of the formula R³L in which R³ is as elsewhere herein and L is a leaving group. The reaction is carried out under standard amide alkylating conditions (Luh, T.; Fung S. H. Synth. Commun. 1979, 9, 757) in an inert solvent at a reaction temperature of 20° C. to 100° C. Appropriate bases include sodium or sodium hydride and are usually employed in molar excess. Suitable solvents include tetrahydrofuran or N,N-dialkylformamides such as N,N-dimethylformamide.

Alternatively, alkylation may be accomplished via phase-transfer catalyst (PTC) techniques. Such techniques comprise carrying out the reaction in the presence of catalyst and in a liquid-liquid two phase solvent system (Gajda, T.; Zwierzak, A. Synthesis, Communications 1981, 1005), or preferably, in a solid-liquid system (Yamawaki, J.; Ando, T.; Hanafusa, T. Chem, Lett. 1981, 1143; Koziara, A.; ZaWasZki, S; Zwierzak, A. Synthesis 1979, 527, 549). A liquid-liquid two-phase system is comprised of an aqueous phase consisting of a concentrated alkali hydroxide solution (e.g., 50% aqueous sodium hydroxide), an organic phase comprised of an inert water-immiscible organic solvent solvent, and an appropriate catalyst. A solid-liquid system consists of a powdered alkali hydroxide/alkali carbonate suspended in an organic solvent and catalyst.

The reaction is effected by adding slowly to a PTC system containing a compound of Formula V an alkylating agent of the formula R³L until 10 to 50% in excess. The reaction mixture is kept at reflux until the reaction is complete. The mixture is then cooled to room temperature and the compound of Formula I is isolated by conventional methods. Suitable organic solvents include benzene, toluene, and the like. Appropriate catalysts include alumina coated with potassium fluoride and quaternary ammonium sulfates such as tetra-n-butyl-ammonium hydrogen sulfate and tricaprylylmethylammonium chloride.

A variation of Scheme II comprises converting a compound of Formula XIa to a compound of Formula XI by one of the above described alkylation processes and then proceeding as in Step 2 of Scheme I to form a compound of Formula I.

Additional Processes

Compounds of Formula I in which R³ is XIV (compounds of Formula I in which the cyclic amine portion of R³ is in the N-oxide form) may be prepared by oxidation of the corresponding compound of Formula I in which R³ is XIV, preferably the nonsalt form. The oxidation is carried out at a reaction temperature of approximately 0° C. with an appropriate oxidizing agent and in a suitable inert, organic solvent. Suitable oxidizing agents include peroxy acids such as trifluoroperacetic acid, permaleic acid, perbenzoic acid, peracetic acid, and m-chloroperoxybenzoic acid. Suitable solvents include halogenated hydrocarbons, e.g., dichloromethane. Alternatively, the compounds of Formula I in which R³ is XIV may be prepared using N-oxide derivatives of the starting materials or intermediates, which may be prepared in a similar manner.

Compounds of Formula I in which R³ is XIII (compounds of Formula I wherein the cyclic amine portion of R³ is not in the N-oxide form) are also prepared by reduction of the corresponding compound of Formula I in which R³ is XIV. The reduction is carried out under standard conditions with an appropriate reducing agent in a suitable solvent. The mixture is occasionally agitated while the reaction temperature is gradually increased over a range of 0° C. to 80° C. Appropriate reducing agents include sulfur, sulfur dioxide, triaryl phosphines (e.g., triphenyl phosphine), alkali borohydrides (e.g., lithium borohydride, sodium borohydride, etc.), phosphorus trichloride and tribromide. Suitable solvents include acetonitrile, ethanol or aqueous diozane.

As will be apparent to one of ordinary skill in the art, compounds of Formula I may be prepared as individual isomers or mixtures of isomers. Isomers which are diastereomers have distinct physical properties (e.g., melting points, boiling points, solubilities, reactivity, etc.) and are readily separated by taking advantage of these dissimilarities. For example, diastereomers can be separated by chromatography or, preferably, by separation/resolution techniques based upon differences in solubility.

Optical isomers can be separated by reacting the racemic mixture with an optically active resolving agent to form a pair of diastereomeric compounds. The isomers are then separated by any of the techniques described above for the separation of diastereomers and the pure optical isomer recovered, along with the resolving agent, by any practical means that would not result in racemization. While resolution of optical isomers can be carried out using covalent diastereomeric derivatives of compounds of Formula II, dissociable complexes are preferred, e.g., crystalline diastereomeric salts. Suitable resolving acids include tartaric acid, o-nitrotartranilic acid, mandelic acid, malic acid, the 2-arylpropionic acids in general, and camphorsulfonic acid.

Individual isomers of compounds of Formula I can also be separated by such methods as direct or selective crystallization or by any other method known to one of ordinary skill in the art. A more detailed description of the techniques applicable to the resolution of stereoisomers of compounds of Formula I can be found in Jean Jacques; Andre Collet; Samuel H. Wilen Enantiomers, Racemates and Resolutions 1981, John Wiley & Sons, Inc. Alternatively, individual isomers of compounds of Formula II can be prepared using the isomeric forms of the starting materials.

Compounds of Formula I can be converted to the corresponding acid addition salt with a pharmaceutically acceptable inorganic or organic acid. Inorganic and organic acids and bases suitable for the preparation of the pharmaceutically acceptable salts of compounds of Formula I are set forth in the definitions section of this application.

Compounds of Formula I in the acid addition salt form are converted to the corresponding free base by treatment with a suitable base such as ammonium hydroxide solution, sodium hydroxide or the like.

Of the two processes for synthesizing compounds of Formula II described within this application, Scheme I is preferred. While compounds of Formula II may be synthesized by the process described in Scheme II, the alkylation step therein may require severe reaction conditions and is usually restricted to alkylation of unsubstituted amides with primary alkylating agents, e.g., CH₃L.

In summary, the process for preparing the compounds of Formula I is:

(1) reacting a compound of Formula XI with a formylating agent in the presence of a strong base and then acidifying to form a compound of Formula XII or reacting a compound of Formula XIIa with an alkylating agent of the formula R³L to form a compound of Formula I;

(2) optionally hydrogenating a compound of Formula XII to form a compound of Formula I,

(3) optionally reacting with or exchanging substituents present on a compound of Formula II to form an additional substituted compound of Formula I;

(4) optionally converting a salt of a compound of Formula I to the corresponding compound of Formula I;

(5) optionally converting a compound of Formula I to a corresponding pharmaceutically acceptable salt;

(6) optionally oxidizing a compound of Formula I in which R³ is XIII to form the corresponding N-oxide;

(7) optionally reducing the N-oxide of a compound of Formula I to the corresponding compound of Formula I in which R³ is XIII; or

(8) optionally separating a mixture of isomers of a compound of Formula I into a single isomer.

Another process of this invention is depicted below in reaction scheme III:

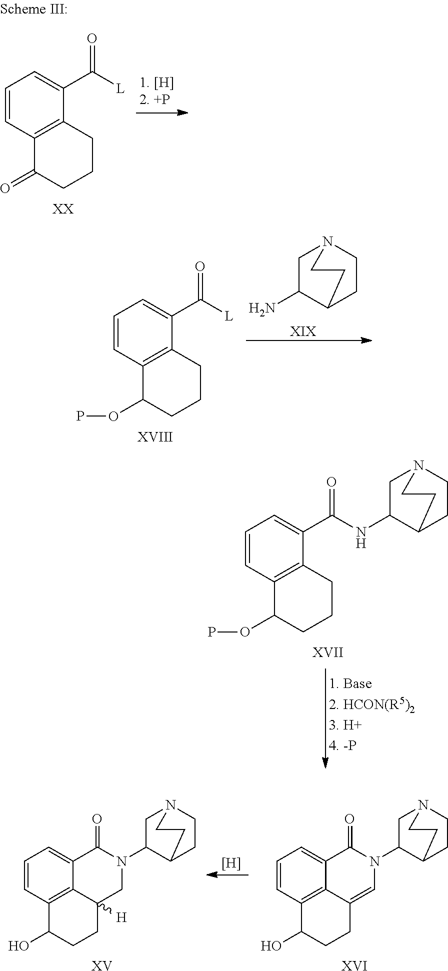

in which L is a leaving group and R⁵ is (C1-C4)alkyl.

A person skilled in the are would recognize that the protected alcohol in scheme III can be positioned at the 4, 5, or 6 position. A person skilled in the art would recognize that Formula XX could have one or more ketones it the structures in the 4, 5, or 6 position.

For example a diastereomeric mixture of 2-(1′-azabicyclo[2.2.2]oct-3′-yl)-6-hydroxy-2,3,3a,4,5,6-hexahydro-1H-benz[de]isoquinolin-1-one XV is prepared by hydrogenating 2-(1′-azabicyclo[2.2.2]oct-3′-yl)-6-hydroxy-2,4,5,6-tetrahydro-1H-benz[de]isoquinolin-1-one XVI. The hydrogenation can be carried out by any means which hydrogenates at the 3- and 3a-positions without dehydroxylating at the 6-position. Such a means can comprise hydrogenating in the presence of a suitable catalyst (e.g., 10% palladium on carbon (10% Pd/C), 5% palladium on barium sulfate (5% Pd/BaSO₄), 5% palladium on alumina (5% Pd/Al₂O₃), 10% palladium on strontium carbonate (10% Pd/SrCO₃), etc., preferably 5% Pd/BaSO₄) and in a suitable organic solvent, typically an ether, alcohol, carboxylic acid, ester, amide or aromatic hydrocarbon and preferably an alcohol (e.g., tetrahydrofuran (THF), ethanol, acetic acid, ethyl acetate, N,N-dimethylformamide (DMF), toluene, etc., preferably ethanol.), at 10° to 78° C., typically at 15° to 30° C. and preferably at approximately 20° C., and at 0 to 200 psig, typically 0 to 100 psig and preferably at approximately atmospheric pressure, and requires 24 to 80 hours.

The compound of Formula XVI is prepared by reacting protected N-(1′-azabicyclo[2.2.2]oct-3′S-yl)-5-hydroxy-5,6,7,8-tetrahydro-1-naphthalenecarboxamide (Formula XVII) with 1 to 20 molar equivalents, typically 1 to 10 molar equivalents and preferably approximately 3 molar equivalents, of a dialkylformamide, typically a di(C1-C4)alkylformamide and preferably DMF, acidifying and then deprotecting. The reaction with the formamide is carried out in the presence of a strong base, typically sodium hydride or an alkyllithium base and preferably butyllithium (e.g., sec-butyllithium, n-butyllithium, etc., preferably sec-butyllithium), and in a suitable solvent, typically an ether (e.g., diethyl ether, dimethoxyethane, tetrahydrofuran (THF), etc., preferably THF), under an inert atmosphere (e.g., nitrogen or argon) at −20° to −75° C., typically at −65° to −75° C. and preferably at approximately −74° C., and requires 0.5 to 5 hours. The reaction mixture is then warmed to between 0° and 30° C., typically to between 15° and 25° C. and preferably to approximately 20° C., and excess molar equivalents of acid, typically 5 to 15 molar equivalents of acid and preferably approximately 10 molar equivalents of hydrochloric acid, is added and the acidified mixture is stirred for 2 to 5 hours.

The deprotection can be carried out by any means which removes the protective group to give the desired unprotected product in reasonable yield. For example, a convenient deprotecting method, particularly when the protective group is tert-butyldiphenylsilyl comprises reacting the protected compound with tetrabutylammonium fluoride in a suitable solvent, typically an ether and preferably THF. The deprotection is carried out in suitable organic solvent at 0° to 50° C., typically at 15° to 25° C. and preferably at approximately 20° C., and requires 1 to 24 hours. A detailed description of the techniques applicable to protective groups and their removal can be found in Greene, T. W.; Protective Groups in Organic Synthesis 1981; John Wiley & Sons, Inc.

The compound of Formula XVII is prepared by reacting a protected 5-hydroxy-1,2,3,4-tetrahydro-1-naphthoic acid derivative (Formula XVIII) with 1-azabicyclo[2.2.2]oct-3-ylamine (Formula XIX). The reaction is carried out under a nitrogen atmosphere in a suitable inert organic solvent, typically an aromatic hydrocarbon, halogenated hydrocarbon or ether and preferably an aromatic hydrocarbon (e.g., toluene, methylene chloride, THF, etc. preferably toluene), at 20° to 200° C., typically at 90° to 130° C. and preferably at approximately 120° C., and requires 10 to 72 hours.

The 1-azabicyclo[2.2.2]oct-3-ylamine is commercially available or can be readily prepared by methods known to those of ordinary skill in the art. The compound of Formula XVIII is prepared by reducing a 5-oxo-1,2,3,4-tetrahydro-1-naphthoic acid derivative (Formula XX) to give a corresponding unprotected 5-hydroxy-1,2,3,4-tetrahydro-1-naphthoic acid derivative and then protecting. The reduction can be effected with a suitable reducing agent, preferably an alkali borohydride (e.g., sodium borohydride, lithium borohydride, etc. preferably sodium borohydride) in a suitable solvent, typically an alcohol (e.g., methanol, ethanol, propanol, isopropanol, etc., preferably ethanol), at −20° to 30° C., typically at −10° to 30° C. and preferably at approximately 0° C., and requires 1 to 5 hours. A suitable protective group can be created by reacting the 5-hydroxy-1,2,3,4-tetrahydro-1-naphthoic acid derivative with 1 to 5 molar equivalents of a suitable protective agent (e.g., tert-butyldiphenylsilyl chloride, tert-butyldimethylsilyl chloride, etc., preferably tert-butyldiphenylsilyl chloride) in a suitable solvent (e.g., DMF, methylene chloride, etc., preferably DMF). For example, a compound of Formula VXIII wherein P is tert-butyldiphenylsilyl is prepared by reacting the unprotected 5-hydroxy-1,2,3,4-tetrahydro-1-naphthoic acid derivative with tert-butyldiphenylsilyl chloride in the presence of imidazole in DMF. The reaction is carried out at 0° to 60° C., typically 0° to 40° C. and preferably at approximately 20° C., and requires 1 to 30 hours.

Compounds of Formula XX in which L is hydroxy or (C1-C4)alkoxy can be prepared by reacting 2-methyl-5,6,7,8-tetrahydro-2H-1-benzopyran-5-one with propiolic acid or (C1-C4) alkyl propiolate, respectively. Preferably the reaction is carried out with ethyl propiolate at 20° to 150° C., typically at 50° to 140° C. and preferably at approximately 115° C. and requires 1 to 5 hours. Other leaving groups can be prepared by treating a compound of Formula XVIII in which L is hydroxy with an appropriate agent (e.g., methanesulfonyl chloride, thionylchloride, phosphorus pentachloride, phosphorus oxychloride, etc.). For example, a compound of Formula XVIII in which L is chloro can be prepared by reacting 5-oxo-5,6,7,8-tetrahydro-1-naphthoic acid with thionyl chloride in a suitable solvent, typically an aromatic hydrocarbon or halogenated hydrocarbon (e.g., toluene, methylene chloride, etc. preferably toluene), at 25° to 50° C., typically at 40° to 50° C. and preferably at approximately 50° C., and requires 1 to 2 hours.

The 2-methyl-5,6,7,8-tetrahydro-2H-1-benzopyran-5-one is prepared by reacting 1,3-cyclohexanedione with crotonaldehyde. The reaction is carried out in a suitable solvent (e.g., pyridine, methylpyridine, 2,4-lutidine, pyrrolidine, etc., preferably pyridine) under an inert atmosphere (e.g., argon or nitrogen) at 100° to 130° C., typically at 110° to 120° C. and preferably at approximately 115° C., and requires 1 to 24 hours.

Depending upon the reaction conditions, isolation/separation techniques and starting materials, the compounds of Formulae XV, XVI, XVII and XIX may be converted to or prepared as their non-salt or salt forms. Thus, the compounds of Formula XV, XVI, XVII and XIX can be utilized in the processes of this invention as a non-salt or salt form in order for the process described to fall within the invention, and the invention includes those processes wherein the compounds are in non-salt form and those processes wherein the compounds are salts. Accordingly, while some forms of the compounds of Formulae XV, XVI, XVII and XIX are preferred, unless indicated otherwise, the description or naming of a particular compound in the specification or in the claims is intended to include both the non-salt form and salt forms, pharmaceutically acceptable or otherwise, thereof.

The compounds of Formulae XV, XVI, XVII and XIX and XVIII each contain one or more chiral centers and can be separated into or prepared as individual stereoisomers and/or mixtures of stereoisomers. Accordingly, while some stereoisomers or mixtures of stereoisomers of the compounds of Formulae XV, XVI, XVII and XIX and XVIII are preferred, unless indicated otherwise, the description or naming of a particular chiral compound in the specification or in the claims is intended to include individual stereoisomers and the mixtures, racemic or otherwise, thereof.

The individual stereoisomers of the compound of Formula XV can be separated from a non-enantiomeric diastereomeric mixture of the compound of Formula XV by chromatography, by separation/resolution techniques based upon differences in solubility, by direct or selective crystallization or by any other method known to one of ordinary skill in the art. For example, 2-(1′-azabicyclo[2.2.2]oct-3′S-yl)-6R-hydroxy-2,3,3aS,4,5,6-hexahydro-1H-benz[de]isoquinoline-1-one is readily prepared from a diastereomeric mixture of 2-(1′-azabicyclo[2.2.2]oct-3′S-yl)-6R-hydroxy-2,3,3a,4,5,6-hexahydro-1H-benz[de]isoquinolin-1-one by silica gel column chromatography.

A non-enantiomeric diastereomeric mixture of the compound of Formula XV can be prepared by reacting an enantiomeric diastereomeric mixture with an optically active acid (e.g., tartaric acid, mandelic acid, malic acid, the 2-arylpropionic acids in general, camphorsulfonic acid, etc.) to form diastereomeric crystalline salts. The non-enantiomeric mixture of crystalline salts is then separated into individual diastereomers by any of the methods described above and the pure diastereomers of the compound of Formula XV are recovered, along with the optically active acid, by any practical means that would not result in racemization. A more detailed description of the techniques applicable to the preparation of stereoisomers can be found in Jean Jacques, Andre Collet, Samuel H. Wilen, Enantiomers, Racemates and Resolutions, John Wiley & Sons, Inc. (1981).

A non-enantiomeric diastereomeric mixture of the compound of Formula XV containing the (6R,3aR,3′S)-, (6S,3aS,3′S)-, (6R,3aS,3′S)- and (6S,3aR,3′S)-diastereomers can be prepared by proceeding as described above and hydrogenating a diastereomeric mixture of 2-(1′-azabicyclo[2.2.2]oct-3′S-yl)-6-hydroxy-2,4,5,6-tetrahydro-1H-benz[de]isoquinolin-1-one. A diastereomeric mixture of the compound of Formula XV containing a mixture of the (6S,3aR,3′S)- and (6S,3aS,3′S)-diastereomers or a mixture of the (6R,3aR,3′S)- and (6R,3aS,3′S)-diastereomers can be prepared by proceeding as described above and hydrogenating 2-(1′-azabicyclo[2.2.2]oct-3′S-yl)-6S-hydroxy-2,4,5,6-tetrahydro-1H-benz[de]isoquinolin-1-one or 2-(1′-azabicyclo[2.2.2]oct-3′S-yl)-6R-hydroxy-2,4,5,6-tetrahydro-1H-benz[de]isoquinolin-1-one, respectively. The individual diastereomers of the compound of Formula XV can then be separated by any of the separation/resolution techniques described above.

The 2-(1′-azabicyclo[2.2.2]oct-3′S-yl)-6-hydroxy-2,4,5,6-tetrahydro-1H-benz[de]isoquinolin-1-one can be prepared as a diastereomeric mixture by proceeding as described above and reacting a diastereomeric mixture of protected N-(1′-azabicyclo[2.2.2]oct-3′S-yl)-5-hydroxy-5,6,7,8-tetrahydro-1-naphthalenecarboxamide with a dialkylformamide in the presence of base, acidifying and then deprotecting. The individual diastereomers of 2-(1′-azabicyclo[2.2.2]oct-3′S-yl)-6-hydroxy-2,4,5,6-tetrahydro-1H-benz[de]isoquinolin-1-one can be prepared from a diastereomeric mixture by any of the applicable separation/resolution techniques described above or by proceeding as described above or from the corresponding individual diastereomer of the protected N-(1′-azabicyclo[2.2.2]oct-3′S-yl)-5-hydroxy-5,6,7,8-tetrahydro-1-naphthalenecarboxamide.

A diastereomeric mixture of protected N-(1′-azabicyclo[2.2.2]oct-3′S-yl)-5-hydroxy-5,6,7,8-tetrahydro-1-naphthalenecarboxamide can be prepared by proceeding as described above and reacting an enantiomeric mixture of the compound of Formula XVIII with (S)-1-azabicyclo[2.2.2]oct-3-ylamine. The individual diastereomers of protected N-(1′-azabicyclo[2.2.2]oct-3′S-yl)-5-hydroxy-5,6,7,8-tetrahydro-1-naphthalenecarboxamide can be prepared from a mixture of the diastereomers by any of the separation/resolution techniques described above or can be prepared by proceeding as described above and reacting an individual enantiomer of the compound of Formula XVIII with (S)-1-azabicyclo[2.2.2]oct-3-ylamine.

The individual enantiomers of the compounds of Formula 5 can be prepared from the individual enantiomers of the corresponding unprotected 5-hydroxy-1,2,3,4-tetrahydro-1-naphthoic acid derivative. The individual enantiomers of the unprotected 5-hydroxy-1,2,3,4-tetrahydro-1-naphthoic acid derivative can be prepared by reacting an enantiomeric mixture with an optically active base to form diastereomeric crystalline salts, separating the diastereomeric salts by chromatography, by separation/resolution techniques based upon differences in solubility, by direct or selective crystallization or by any other method known to one of ordinary skill in the art, and then recovering the pure enantiomers, along with the optically active base, by any practical means that would not result in racemization (e.g., see Enantiomers, Racemates and Resolutions 1981; John Wiley & Sons, Inc. cited above).

Alternatively, the individual enantiomers of the unprotected 5-hydroxy-1,2,3,4-tetrahydro-1-naphthoic acid derivative can be prepare by an enantioselective reduction of the compound of Formula XX. The enantioselective reduction is carried out by proceeding as described above and reducing the compound of Formula 6 in the presence of a suitable chiral auxiliary (e.g., azaoxaborodine) or a selective reducing agent (e.g, chlorodiisopinocampheylborane, lithium tri-sec-butylborohydride, etc.). For example, an unprotected 5-hydroxy-1,2,3,4-tetrahydro-1-naphthoic acid derivative wherein the chiral carbon is in the (R)-configuration can be prepared by proceeding as described above and reducing the compound of Formula XX with diborane in the presence of (S)-1-aza-2-boro-3-oxa-4,4-diphenyl[3.3.0]bicyclooctane. Similarly, an unprotected 5-hydroxy-1,2,3,4-tetrahydro-1-naphthoic acid derivative wherein the chiral carbon is in the (S)-configuration can be prepared by proceeding as described above and reducing the compound of Formula 6 in the presence of (R)-1-aza-2-boro-3-oxa-4,4-diphenyl[3.3.0]bicyclooctane. For a more detailed description of the techniques applicable to the enantioselective reduction of unsymmetrical ketones see Singh, V. K.; Synthesis 1992; 7:605.

(S)-1-Azabicyclo[2.2.2]oct-3-ylamine can be prepared by separating the individual enantiomers from a enantiomeric mixture of the amine by any of the applicable separation/resolution techniques described above. Alternatively, (S)-1-azabicyclo[2.2.2]oct-3-ylamine can be prepared by reacting 1-azabicyclo[2.2.2]oct-3-one with an (R)-α-alkylbenzylamine, preferably (R)-1-phenylethylamine, to give the corresponding (R)—N-(α-alkylbenzyl)-3-(1-azabicyclo[2.2.2]octan)imine, reducing the imine to give the corresponding N-(1R-phenylalkyl)-1-azabicyclo[2.2.2]oct-3S-ylamine and then hydrogenolyzing. The reaction with the (R)-α-alkylbenzylamine is carried out in the presence of lithium oxide in a suitable organic solvent, typically an ether and preferably THF, at 10° to 40° C., typically at 15° to 30° C. and preferably at approximately 20° C., and requires 12 to 84 hours. The reduction of the imine can be carried out by catalytic hydrogenation or with a suitable chemical reducing agent.

Hydrogenation of the imine is carried out in the presence of a suitable catalyst preferably 5% Pt/C, and in a suitable organic solvent, typically an alcohol and preferably ethanol, at 10° to 40° C., typically at 15° to 30° C. and preferably at approximately 20° C., and at 0 to 100 psig, typically at 0 to 50 psig and preferably at approximately 20 psig, and requires 1 to 48 hours. Alternatively, the imine can be reduced with a suitable chemical reducing agent, preferably an alkali borohydride (e.g., sodium borohydride, lithium borohydride, etc., preferably sodium borohydride), in a suitable organic solvent, typically an alcohol and preferably ethanol, at −15° to 50° C., typically at 15° to 30° C. and preferably at approximately 20° C., and requires 15 minutes to 3 hours.

The hydrogenolyzation is effected by hydrogenation the N-(1R-phenylalkyl)-1-azabicyclo[2.2.2]oct-3S-ylamine in the presence of a suitable catalyst (e.g., 10% Pd/C, 20% Pd/C, etc., preferably 10% Pd/C) and in a suitable organic solvent, typically an alcohol and water mixture and preferably 5/1 to 2/1 ethanol/water, at 10° to 40° C., typically at 15° to 30° C. and preferably at approximately 20° C., and at 0 to 100 psig, typically at 0 to 20 psig and preferably at approximately 5 psig, and requires 5 to 48 hours.

### Utility

Compounds of the present invention exhibit utility in treating a broad range of diseases in animals, particularly humans. Examples of diseases that can be treated using these compounds include emesis, gastrointestinal disorders, central nervous system (CNS) disorders, cardiovascular disorders or pain.

Compounds of the present invention can be used in the prevention and treatment of emesis. Causes of such emesis include surgical anesthesia, psychological stress, pregnancy, certain disease states, radiotherapy, radiation poisoning, and toxic substances. Disease states which are known to induce emesis include conditions such as gut obstruction, raised intracranial pressure, acute myocardial infarction, migraine headaches and adrenal crisis. Toxic substances which induce emesis include toxins in the form of abnormal metabolites or abnormal accumulation of natural occurring substances associated with such conditions as hepatic coma, renal failure, diabetic ketoacidosis, hyperthyroid crisis, both hypo- and hyperparathyroidism and Addison's disease. Emesis can also be caused by ingested toxins, e.g., enterotoxins in staphylococcus-contaminated foods, or by drugs administered for therapeutic purposes, e.g., digitalis, emetine and chemotherapeutic agents.

Compounds of the present invention can be of particular value in treating (especially preventing) the emesis induced by radiation poisoning, treatment for cancer with radiotherapy or chemotherapy with cytotoxic agents or drug therapy in general wherein a significant side effect is emesis, e.g., amphotericin B in treating immunosuppressed patients, zidovudine (AZT) in the treatment of AIDS and interleukin in treating cancer.

Compounds of the present invention can be useful as prokinetic agents in the treatment of gastrointestinal diseases, i.e., diseases of the stomach, esophagus and of both the large and small intestines. Examples of specific diseases include, but are not limited to, dyspepsia (e.g., non-ulcer dyspepsia), gastric stasis, peptic ulcer, reflux esophagitis, flatulence, bile reflux gastritis, pseudo-obstruction syndrome, irritable colon syndrome (which may result in chronic constipation and diarrhea), diverticular disease, biliary dysmotility (which may result in sphincter of Oddi dysfunction and “sludge” or microscopic crystals in the gall bladder), gastroparesis (e.g., diabetic, postsurgical or idiopathic), irritable bowel syndrome and retarded gastric emptying. The compounds can also be used as short-term prokinetics to facilitate diagnostic radiology and intestinal intubation. In addition, the compounds can be useful for treating diarrhea, particularly diarrhea induced by cholera and carcinoid syndrome.

Compounds of the present invention also can be useful in treating diseases of the central nervous system. Categories of such diseases include cognitive disorders, psychoses, obsessive/compulsive, and anxiety/depression behavior. Cognitive disorders include attentional or memory deficit, dementia states (including senile dementia of the Alzheimer's type and aging), cerebral vascular deficiency and Parkinson's disease. Psychoses that can be treated using the compounds include paranoia, schizophrenia and autism. Obsessive/compulsive behavior that can be treated using the compounds include eating disorders, e.g., bulimia, a condition in which an abnormal and constant craving for food is present.

Representative, treatable anxiety/depressive states include anticipatory anxiety (e.g., prior to surgery, dental work, etc.), depression, mania, seasonal affective disorder (SAD), and the convulsions and anxiety caused by withdrawal from addictive substances such as opiates, benzodiazapines, nicotine, alcohol, cocaine and other drugs of abuse.

Compounds of the present invention can be useful in the treatment of cardiovascular diseases. Such diseases include arrhythmias and hypertension.

It is thought that 5-HT₃ antagonists prevent certain adverse nervous transmissions and/or prevent vasodilation and are therefore of value for reducing perceived levels of pain. Compounds of the invention can, therefore, be used in treating pain such as that associated with cluster headaches, migraines, trigeminal neuralgia and visceral pain (e.g., that caused by abnormal distension of hollow visceral organs).

In summary, an aspect of this invention is a method for treating an animal, particularly a human, exhibiting a disease involving emesis, a gastrointestinal disorder, a CNS disorder, a cardiovascular disorder, or pain by administering a therapeutically effective amount of a compound of the present invention to such animal.

### Methods of Treatment

In still further embodiments, the invention provides methods of treating emesis by administering one or more of the compounds described herein. The compound is preferably administered shortly before the emesis inducing event (i.e. no more than 2 hours before the event). The emesis may be acute phase emesis (i.e. emesis experienced within about 24 hours of an emesis inducing event), or delayed emesis (i.e. emesis experienced after the acute phase, but within seven, six, five or four days of an emesis inducing event). The emesis may constitute chemotherapy induced nausea and vomiting (“CINV”), from moderately or highly emetogenic chemotherapy, radiation therapy induced nausea and vomiting (“RINV”), or post-operative nausea and vomiting (“PONV”).

Throughout this application, various publications are referenced. The disclosures of these publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1) An isolated compound of formula I: 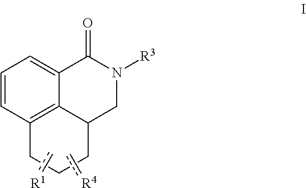
or a pharmaceutically acceptable salt thereof;
wherein R¹ and R⁴ independently are H, hydroxyl, or carbonyl; and
wherein R³ is

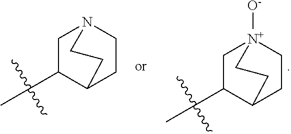

2) (canceled)

3) (canceled)

4) (canceled)

5) (canceled)

6) (canceled)

7) (canceled)

8) (canceled)

9) (canceled)

10) An isolated compound of formula II, III, IV, V, VI, VII, VIII or IX, or a pharmaceutically acceptable salt thereof: 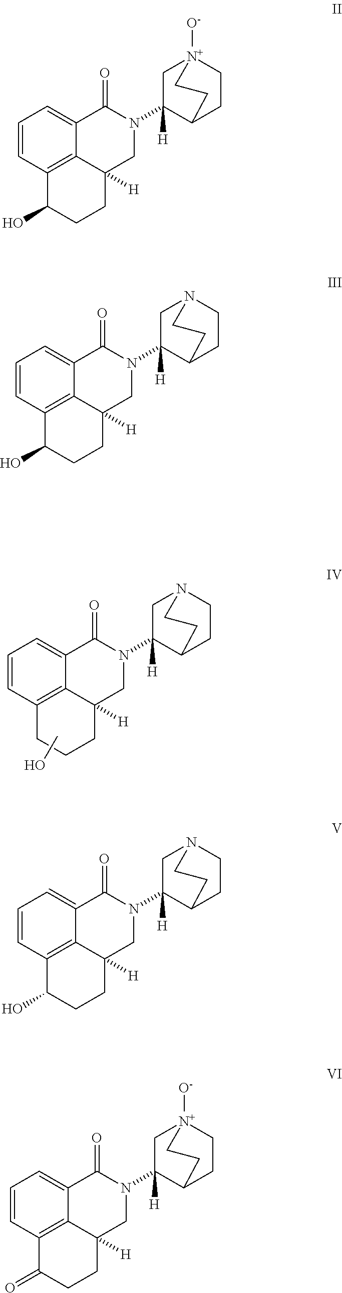 [chemical structure]
